(19) United States
(12) Patent Application Publication
(10) Pub. No.: US 2007/0254900 A1
(43) Pub. Date: Nov. 1, 2007

(54) Treatments for Congestive Heart Failure

(76) Inventors: Ghassan S. Kassab, Zionsville, IN (US); Xiao Lu, Zionsville, IN (US)

Correspondence Address: MOAZZAM & ASSOCIATES, LLC, 7601 LEWINSVILLE ROAD, SUITE 304, MCLEAN, VA, 22102, US

(21) Appl. No.: 11/659,876

(22) PCT Filed: Aug. 11, 2005
(86) PCT No.: PCT/US05/28818

§ 371 Date: Feb. 9, 2007

Related U.S. Application Data
(60) Provisional application No. 60/601,092, filed on Aug. 11, 2004.

(51) Int. Cl. A61K 31/519; A61K 31/03; A61K 31/12; A61P 9/00; A61P 9/04; A61K 31/135; A61K 31/445

(52) U.S. Cl. 514/262.1; 514/315; 514/649; 514/688; 514/754

(57) ABSTRACT

Methods and pharmaceutical preparations for treating heart failure by administering to a human or animal subject a therapeutically effective amount of at least one substance selected from the group consisting of a) SOD mimics (e.g., Tempol), b) NADPH oxidase inhibitors (e.g., Apocynin) and c) other substance that inhibit or reduce the amount of superoxide present in the affected tissues (e.g., the subjects heart and/or blood vessels) and/or increase levels of nitric oxide.

### RELATED APPLICATION

This patent application claims priority to U.S. Provisional Patent Application No. 60/601,092 filed on Aug. 11, 2004, which is expressly incorporated herein by reference.

### BACKGROUND OF THE INVENTION

Nitric oxide (NO) is a compound that is produced by healthy endothelial cells, such as those that line human blood vessels, and is associated with relaxation and dilation of blood vessels. Nitric oxide acts as a signaling molecule in the cardiovascular system. A signaling molecule is a molecule that produces or induces the production of another substance, called a second messenger. The second messenger then brings about some physiologic effect.

When nitric oxide (the signaling molecule)enters a cell, it activates an enzyme called guanylate cyclase, which then causes production of cyclic GMP (the second messenger). The cyclic GMP then causes relaxation and dilation of the blood vessels. In addition to relaxing and dilating blood vessels, nitric oxide also prevent coronary artery disease and strokes by preventing platelets and white blood cells from sticking to the vessel wall. Nitric oxide may also, under certain conditions, reduce the presence of free radicals, which can cause your vessels to age rapidly. Also, nitric oxide suppresses abnormal growth of vascular smooth muscle cells as is known to occur in certain types of atherosclerosis and during reocclusion following balloon angioplasty procedures.

Hypercholesterolemia reduces nitric oxide bioavailability, which in turn results in reduced endothelium-dependent vascular relaxation, and also induces the expression of vascular adhesion molecules and infiltration of inflammatory cells. It has been reported that gene therapy with Nitric Oxide synthase in hypercholesterolemic rabbits substantially reverses the deficit in vascular relaxation exhibited by those animals.

### SUMMARY OF THE INVENTION

Applicants have recently discovered that reversing of blood flow in an artery results in decreased nitric oxide production and that such decrease in nitric oxide production is mediated through an increase in superoxides production. Lu, Xiao and Kassab, Ghassan S., Nitric Oxide is Significantly Reduced During Reverse Flow Because of Increased Superoxide Production; J. Physiology, Vol. 561 (2), Pages 575-582 (2004). Flow reversal, which is known to occur in CHF, is believed to increase in superoxide production and reduce nitric oxide levels in large vessels. In peripheral vessels, CHF will lead to reduction in flow and similar increase in superoxides. Accordingly, the present invention provides treatments for congestive heart failure (CHF) based on inhibition or reduction of superoxides and/or increasing nitric oxide.

In accordance with the invention, there is provided a method for treating heart failure in a human or animal subject, such method comprising the step of administering to the subject, in an amount that is therapeutically effective to increase the concentration of nitric oxide in blood vessels of the heart, at least one substance selected from the group consisting of a) SOD mimics, b) NADPH oxidase inhibitors and c) substance that inhibits the effect of superoxide and/or reduces the amount of superoxide and/or increases the amount of nitric oxide present in the affected tissues (e.g., the subject's blood vessels and/or heart). For example, superoxide is produced in the cell through various pathways. These pathways are mitochondrial oxidase, xanthine oxidase (XO), uncoupled NO synthases, cytochrome P-450 enzymes, and NADPH oxidases. In addition, enzymes such as lipoxygenases may also generate superoxide. In particular, both the NADPH oxidase pathway and the XO pathway have been implicated in endothelial dysfunction in artherosclerosis and heart failure. Inhibitors of these pathways include, but are not limited to; NADPH Oxidase inhibitors such as Apocynin and 4-(2-aminoethyl)-benzenesulfonyl fluoride (AEBSF); 2. Xanthine oxidase inhibitors such as allopurinol or oxypurinol and 3. other oxidase inhibitors, such as Diphenyleneiodonium (DPI) which inhibits NADPH oxidase, XO, nitric oxide synthase, cytochrome P-450 reductase, and mitochondrial oxidase. Further in accordance with the invention, there are provided pharmaceutical preparations for the treatment of CHF, such preparations comprising a) at least one substance selected from the group consisting of i) SOD mimics, ii) NADPH oxidase inhibitors and iii) substances that inhibit superoxide or reduce the amount of superoxide and/or increase the amount of nitric oxide present in affected tissues (e.g., the subjects blood vessels and/or heart) in combination with b) at least one solvent, carrier, vehicle, medium, diluent or excipient agent.

Further aspects, elements and details of the invention will be understood by those of skill in the art upon reading of the detailed description set forth below.

### BRIEF DESCRIPTION OF THE DRAWINGS

FIG. 1 is a graph showing production of NO metbolite vs. flowrate (forward and reverse) in ex vivo porcine femoral artery preparations with and without tempol.

FIG. 2 shows micrographs obtained by laser confocal microscopy showing the concentrations of superoxide in carotid artery wall under control (forward flow), reverse flow (FR) and reverse flow+Apocynin (AC) conditions.

### DETAILED DESCRIPTION AND EXAMPLES

The present invention provides methods for treating a cardiovascular disorders such as congestive heart failure (CHF) in human or animal subjects by administering to the subject a therapeutically effective amount of a substance that mimics the action of superoxide dismutase (SOD) (hereinafter referred to as “SOD mimics”) and/or any other substance that decreases production of, scavanges, blocks the effects of, reduces the amount of or otherwise inhibits superoxide and/or increases the concentration of nitric oxide.

There are two major classes of SOD mimics, those that contain metals and those that are metal-independent. The three metals contained in complexes normally studied are copper, iron, and manganese. Metal-independent SOD mimics are various nitroxides complexes. SOD mimics catalyze the dismutation of O2 to hydrogen peroxide (H2O2) and dioxygen (O2). Examples of SOD mimics useable in this invention include those described or disclosed in U.S. Pat. No. 6,180,620 (Salvemini); U.S. Pat. No. 6,214,817 (Riley et al.); U.S. Pat. No. 6,245,758 (Salvemini); U.S. Pat. No. 6,395,725 (Salvemini); Published United States Patent Application No. US2002/0072512 A1 (Salvemini); Published United States Patent Application No. US2002/0128248 A1 (Salvemini); Published United States Patent Application No. US2004/0132706 A1 (Salvemini) and Salvemini, D., et el., Therapeutic Potential of Superoxide Dismutase Mimetics as Therapeutic Agents in Critical Care Medicine, Crit Care Med 2003, Vol. 31, Vol. 1, each of the aforementioned patents and patent applications being expressly incorporated herein by reference.

The SOD mimics used in the present invention may have the general formula 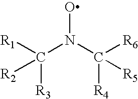
Wherein,
R₁, R₂, R₅ and R₆ are each independently C₁-C₂₀ alkyl, C₂-C₂₀ alkenyl or C₂-C₂₀ alkynyl; or R₁ and R₂ and/or R₅ and R₆ combine, with the linking atom, to form a C₃-C₁₂ cyclic ring;
R₃ and R₄ may combine, including the nitrogen atom (N), to form a heterocyclic 5-7 member ring structure, wherein (i) at least one ring methylene group is replaced by a heteroatom selected from NR, O and S or an oxo (C═O) group, where R is selected from H, lower alkyl, lower alkenyl, lower alkynyl; and (ii) at least one methylene group hydrogen is substituted with a group selected from C₁-C₂₀ alkyl, C₂-C₂₀ alkenyl, C₂-C₂₀ alkynyl, —OH, —O—CO—R₇, —O—R₇, —S—R₇ and —NR₈R₉, where R₇ is selected from C₁-C₂₀ alkyl, C₂-C₂₀ alkenyl, C₂-C₂₀ alkynyl, aralkyl and glycosyl; R₈ and R₉ are selected independently from H, C₁-C₂₀ alkyl, C₂-C₂₀ alkenyl, C₂-C₂₀ alkynyl, aryl, aralkyl, glycosyl and —CO—R₁₀, where R₁₀ is lower alkyl or cycloalkyl.

One commercially available SOD mimic substance that is useable in the present invention is Tempol (4-hydroxy-2,2,6,6-tetramethyl-4-piperidine-N-oxyl). Tempol is commercially available from Sigma Chemical, St. Louis, Mo. To treat CHF in a human patient, tempol may be administered at a dose of from about 2 to about 200 mg/kg or from about 5 to about 55 mg/kg, i.v., or by other routes of administration at doses that are higher or lower than 5 to about 55 mg/kg. It will be appreciated that any effective dosages and/or routes of administration and/or dosing schedules may be employed.

The potential utility of tempol and other SOD mimics in the treatment of CHF has been established based on laboratory testing, as described in Example 1 below:

### EXAMPLE 1

### The Effects of Tempol On Superoxide Production During Flow Reversal

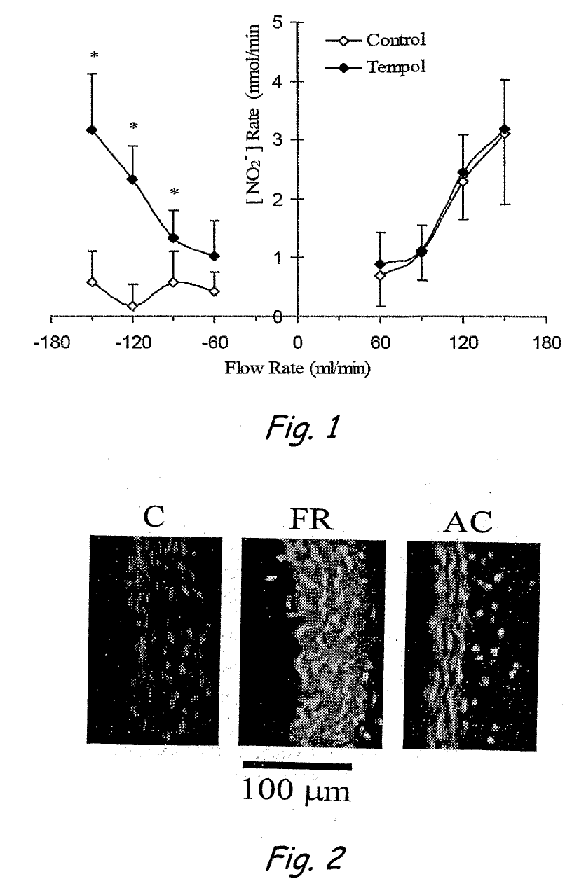

A purfusate was passed through an in vitro porcine femoral artery preparation in the forward and reverse flow directions and the levels of NO metabolite (nitrite) were measured at various flowrates in the forward and reverse directions. Thereafter, tempol was added to the perfusate and the levels of NO metabolite (nitrite) were again measured at various flowrates in the forward and reverse directions. The addition of tempol to the perfusate did not cause a statistically significant increase in NO production during forward flow. However, the addition of tempol did significantly increase NO production during reverse flow. These data (mean ±SD ) are shown graphically in FIG. 1. The asterisk denotes statistical significance by ANOVA analysis between groups at the respective flows (P<0.05).

Another category of compounds that may be used in accordance with the present invention are oxidase blockers such as NADPH oxidase inhibitors. One such NADPH oxidase inhibitor is Apocynin (4-hydroxy-3-methoxy-acetophenone). Other NADPH Oxidase inhibitors include, for example, 4-(2-aminoethyl)-benzenesulfonyl fluoride (AEBSF) and Diphenyleneiodonium (DPI). The potential utility of Apocynin and other NADPH Oxidase inhibitors in the treatment of CHF has been established based on laboratory testing, as described in Example 2 below:

### EXAMPLE 2

### The Effect of Apocynin On Superoxide Concentration In Arterial Wall During Flow Reversal

The carotid arteries of animals were removed and examined by laser confocal microscopy excitation (514 nm;

emission, 605 nm; Objective, 40x. C.3.a. Visualization of NO and O2—Using Fluorescence) following perfusion under conditions of: Control (C), Flow Reversal (FR), and Apocynin+Flow Reversal (AC). The apocynin was administered in drinking water (1 mM) to an animal concurrently with carotid flow reversal. Dihydroethidine (DHE), a fluorescent dye, was used to indicate superoxide in arterial wall. As seen in FIG. 2, the animal that received Apocynin+Flow Reversal clearly had less superoxide present in the artery wall than the animal that received Flow Reversal without Apocynin. Thus, the administration of apocynin reduced the amount of superoxide present under reverse flow conditions.

The dosages of Apocynin useable to treat CHF are in the range of from about 2 mg/kg to about 200 mg/kg and more preferably in the range of from about 4 mg/kg to about 40 mg/kg, i.v., or by other routes of administration and/or at other dosages. It will be appreciated that any effective dosages and/or routes of administration and/or dosing schedules may be employed.

It is to be appreciated that, although specific examples are provided above with respect to only Tempol and Apocynin, such examples also generally demonstrate the utility of any therapy that inhibits superoxide or reduces the amount of superoxide and/or increases the amount of nitric oxide present in affected tissues (e.g., the subjects blood vessels and/or heart)

It is to be appreciated that the invention has been described hereabove with reference to certain examples or embodiments of the invention but that various additions, deletions, alterations and modifications may be made to these examples and embodiments without departing from the intended spirit and scope of the invention. For example, any element or attribute of one embodiment or example may be incorporated into or used with another embodiment or example, unless to do so would render the embodiment or example unsuitable for its intended use. All reasonable additions, deletions, modifications and alterations are to be considered equivalents of the described examples and embodiments and are to be included within the scope of the following claims.

What is claimed is:

1. A method for treating heart failure in a human or animal subject, said method comprising the step of:
administering to the subject, in an amount that is therapeutically effective to increase the concentration of nitric oxide in blood vessels of the heart, at least one substance selected from the group consisting of a) SOD mimics, b) NADPH oxidase inhibitors and c) substances that inhibit the effect of superoxide, reduce the amount of superoxide or increase the amount of nitric oxide.

2. A method according to claim 1 wherein the substance comprises an SOD mimic.

3. A method according to claim 1 wherein the substance comprises an NADPH oxidase inhibitor.

4. A method according to claim 1 wherein the substance comprises an SOD mimic that is metal independent.

5. A method according to claim 1 wherein the substance comprises an SOD mimic that contains metal.

6. A method according to claim 1 wherein the substance comprises tempol (4-hydroxy-2,2,6,6-tetramethyl-4-piperidine-N-oxyl).

7. A method according to claim 4 wherein the tempol is administered at a dose of from about 2 mg/kg to about 200 mg/kg.

8. A method according to claim 4 wherein the tempol is administered at a dose of from about 5 mg/kg to about 55 mg/kg.

9. A method according to claim 1 wherein the substance comprises an SOD mimic having the general formula: 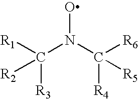
wherein,
R₁, R₂, R₅ and R6 are each independently C₁-C₂₀ alkyl, C₂-C₂₀ alkenyl or C₂-C₂₀ alkynyl; or R₁ and R₂ and/or R₅ and R₆ combine, with the linking atom, to form a C₃-C₁₂ cyclic ring;
R₃ and R₄ may combine, including the nitrogen atom (N), to form a heterocyclic 5-7 member ring structure, wherein (i) at least one ring methylene group is replaced by a heteroatom selected from NR, O and S or an oxo (C═O) group, where R is selected from H, lower alkyl, lower alkenyl, lower alkynyl; and (ii) at least one methylene group hydrogen is substituted with a group selected from C₁-C₂₀ alkyl, C₂-C₂₀ alkenyl, C₂-C₂₀ alkynyl, —OH, —O—CO—R₇, —O—R₇, —S—R₇ and —NR₈R₉, where R₇ is selected from C₁-C₂₀ alkyl, C₂-C₂₀ alkenyl, C₂-C₂₀ alkynyl, aralkyl and glycosyl; R₈ and R₉ are selected independently from H, C₁-C₂₀ alkyl, C₂-C₂₀ alkenyl, C₂-C₂₀ alkynyl, aryl, aralkyl, glycosyl and —CO—R₁₀, where R₁₀ is lower alkyl or cycloalkyl.

10. A method according to claim 1 wherein the substance comprises Apocynin.

11. A method according to claim 10 wherein the Apocynin is administered at a dose of from about 2 mg/kg to about 200 mg/kg.

12. A method according to claim 10 wherein the Apocynin is administered at a dose of from about 4 mg/kg to about 40 mg/kg.

13. A method according to claim 1 wherein the substance comprises an NADPH Oxidase inhibitor.

14. A method according to claim 1 wherein the NADPH Oxidase inhibitor comprises at least one compound selected from the group consisting of Apocynin, Diphenyleneiodonium (DPI) and 4-(2-aminoethyl)-benzenesulfonyl fluoride (AEBSF).

15. A method according to claim 1 wherein the substance comprises an Xanthine Oxidase inhibitor.

16. A method according to claim 1 wherein the Xanthine Oxidase inhibitor comprises at least one compound selected from the group consisting of allopurinol or oxypurinol.

17. A method according to claim 1 wherein the substance comprises at least one compound selected from the group consisting of NADPH oxidase inhibitors, XO inhibitors, nitric oxide synthase inhibitors, cytochrome P-450 reductase inhibitors, and mitochondrial oxidase inhibitors.

18. A method according to claim 17 wherein the substance is selected from the group consisting of: Tempol, Apocynin, Diphenyleneiodonium (DPI), 4-(2-aminoethyl)-benzenesulfonyl fluoride (AEBSF), allopurinol and oxypurinol.

19. A pharmaceutical preparation for the treatment of heart failure in human or animal subjects, said preparation comprising:
a) at least one substance selected from the group consisting of a) SOD mimics, b) NADPH oxidase inhibitors and c) substance that inhibit the production of superoxide or reduce superoxide levels in the subject; and
b) at least one solvent, carrier, diluent or excipient agent.

20. The use, in the manufacture of a pharmaceutical for administration to a human or animal subject for the treatment of heart failure, of at least one substance selected from the group consisting of a) SOD mimics, b) NADPH oxidase inhibitors and c) substances that inhibit the production of superoxide, reduce superoxide levels or increase nitric oxide levels in affected tissue of the subject.

21. A use according to claim 20 wherein the substance comprises a metal independent SOD mimic.

22. A use according to claim 20 wherein the substance comprises a metal containing SOD mimic.

23. A use according to claim 20 wherein the substance comprises tempol (4-hydroxy-2,2,6,6-tetramethyl-4-piperidine-N-oxyl).

24. A use according to claim 20 wherein the substance comprises an SOD mimic having the general formula: 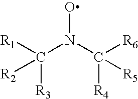
wherein,
R₁, R₂, R₅ and R₆ are each independently C₁-C₂₀ alkyl, C₂-C₂₀ alkenyl or C₂-C₂₀ alkynyl; or R₁ and R₂ and/or R₅ and R₆ combine, with the linking atom, to form a C₃-C₁₂ cyclic ring;
R₃ and R₄ may combine, including the nitrogen atom (N), to form a heterocyclic 5-7 member ring structure, wherein (i) at least one ring methylene group is replaced by a heteroatom selected from NR, O and S or an oxo (C═O) group, where R is selected from H, lower alkyl, lower alkenyl, lower alkynyl; and (ii) at least one methylene group hydrogen is substituted with a group selected from C₁-C₂₀ alkyl, C₂-C₂₀ alkenyl, C₂-C₂₀ alkynyl, —OH, —O—CO—R₇, —O—R₇, —S—R₇ and —NR₈R₉, where R₇ is selected from C₁-C₂₀ alkyl, C₂-C₂₀ alkenyl, C₂-C₂₀ alkynyl, aralkyl and glycosyl; R₈ and R₉ are selected independently from H, C₁-C₂₀ alkyl, C₂-C₂₀ alkenyl, C₂-C₂₀ alkynyl, aryl, aralkyl, glycosyl and —CO—R₁₀, where R₁₀ is lower alkyl or cycloalkyl.

25. A use according to claim 20 wherein the substance comprises an NADPH Oxidase inhibitor.

26. A use according to claim 25 wherein the NADPH Oxidase inhibitor comprises at least one compound selected from the group consisting of Apocynin, Diphenyleneiodonium (DPI) and 4-(2-aminoethyl)-benzenesulfonyl fluoride (AEBSF).

27. A use according to claim 20 wherein the substance comprises an Xanthine Oxidase inhibitor.

28. A use according to claim 27 wherein the Xanthine Oxidase inhibitor comprises at least one compound selected from the group consisting of allopurinol or oxypurinol.

29. A use according to claim 20 wherein the substance comprises at least one compound selected from the group consisting of NADPH oxidase inhibitors, XO inhibitors, nitric oxide synthase inhibitors, cytochrome P-450 reductase inhibitors, and mitochondrial oxidase inhibitors.

30. A use according to claim 29 wherein the substance comprises at least one compound selected from the group consisting of: Tempol, Apocynin, Diphenyleneiodonium (DPI), 4-(2-aminoethyl)-benzenesulfonyl fluoride (AEBSF), allopurinol and oxypurinol.
